(19) United States
(12) Patent Application Publication
(10) Pub. No.: US 2021/0139550 A1
(43) Pub. Date: May 13, 2021

(54) HINGES 1 AND/OR 4 MODIFIED DYSTROPHINS FOR DYSTROPHINOPATHY THERAPY

(71) Applicant: CURATORS OF THE UNIVERSITY OF MISSOURI, Columbia, MO (US)

(72) Inventors: Dongsheng DUAN, Columbia, MO (US); Lakmini WASALA, Columbia, MO (US); Yi LAI, Columbia, MO (US); Yongping YUE, Columbia, MO (US)

(21) Appl. No.: 17/045,266

(22) PCT Filed: Apr. 3, 2019
(86) PCT No.: PCT/US2019/025496

§ 371 Date: Oct. 5, 2020

Related U.S. Application Data
(60) Provisional application No. 62/651,772, filed on Apr. 3, 2018.

(51) Int. Cl. C07K 14/47; C12N 15/86; A61K 48/00; A61K 38/17

(52) U.S. Cl. CPC C07K 14/4708; C12N 15/86; C12N 2830/008; A61K 38/1719; C12N 2750/14143; A61K 48/0058

(57) ABSTRACT

Disclosed are compositions and methods for treating dystrophinopathies. Compositions include modified dystrophin polynucleotides that encode modified dystrophin proteins having modified hinge 1 (H1) and/or hinge 4 (H4). Also disclosed are methods for treating dystrophinopathies by administering compositions encoding modified dystrophin proteins having modified hinge 1 (H1) and/or modified hinge 4 (H4).

### CROSS REFERENCE TO RELATED APPLICATIONS

This application claims priority to U.S. Provisional Application Serial No. 62/651,772, filed on Apr. 3, 2018, the disclosure of which is hereby incorporated by reference in its entirety.

### STATEMENT REGARDING FEDERALLY SPONSORED RESEARCH OR DEVELOPMENT

This invention was made with government support under NS090634 awarded by the National Institutes of Health. The government has certain rights in the invention.

### STATEMENT IN SUPPORT FOR FILING A SEQUENCE LISTING

A paper copy of the Sequence Listing and a computer readable form of the Sequence Listing containing the file named “18UMC046_ST25.txt”, which is 151,883 bytes in size (as measured in MICROSOFT WINDOWS® EXPLORER), are provided herein and are herein incorporated by reference. This Sequence Listing consists of SEQ ID NOs:1-18.

### BACKGROUND OF THE DISCLOSURE

The present disclosure is directed to compositions and methods for treating dystrophinopathies. Compositions include modified dystrophin polynucleotides that encode modified dystrophin proteins having modified hinge 1 (H1) and/or hinge 4 (H4). Also disclosed are methods for treating dystrophinopathies by administering compositions encode modified dystrophin proteins having modified hinge 1 (H1) and/or modified hinge 4 (H4).

Dystrophinopathy refers to a group of diseases caused by mutations in the dystrophin gene. Dystrophinopathy includes Duchenne muscular dystrophy (DMD), Becker muscular dystrophy (BMD), X-linked dilated cardiomyopathy (XLDC) and their carriers.

The dystrophin gene (2.4 mb) is one of the largest genes in the genome. The dystrophin mRNA is 14 kb. The dystrophin protein consists of four regions; amino terminus (NT), central rod domain with 24 spectrin-like repeat (R) regions, and four hinge (H) regions, cysteine-rich domain (CR), and carboxyl-terminal (CT) domain. While the biological functions of NT, CR and CT as well as repeats in the rod domain and H2 and H3 have been extensively interrogated, little is known about the function of H1 and H4s.

Replacing the mutated dystrophin gene with a functional one by viral or non-viral mediated gene delivery is a highly promising strategy to treat dystrophinopathy. The dystrophin gene and its cDNA are too big for packaging into viral vectors, in particular, the adeno associated virus (AVV) vector. For this reason, there has been an enormous interest in developing synthetic mini and microgenes. Of particular importance is AAV micro-dystrophin gene therapy. Several clinical trials have been initiated using the AAV microgene vector. Strategies that can improve the current dystrophin microgenes can be beneficial for treating dystrophinopathies.

Accordingly, there exists a need for alternative dystrophin microgenes and methods for treating dystrophinopathies.

### BRIEF DESCRIPTION OF THE DISCLOSURE

In one aspect, the present disclosure is directed to a nucleic acid encoding a modified dystrophin comprising a hinge region modification to at least one of hinge 1 (H1) region, hinge 4 (H4) region, and combinations thereof.

In one aspect, the present disclosure is directed to a vector comprising a nucleic acid encoding a modified dystrophin comprising a hinge region modification to at least one of hinge 1 (H1) region, hinge 4 (H4) region, and combinations thereof.

In one aspect, the present disclosure is directed to a method for treating a dystrophinopathy in a subject in need thereof. The method includes administering a modified dystrophin having a modified hinge region to a subject in need thereof.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a schematic illustrating the protein structure of pYL90 (plasmid with full-length microdystrophin) and pLW₁ (plasmid with ΔH₁ microdystrophin).

FIGS. 2A & 2B are Western blots detecting expression of AAV virus LW₁-treated (FIG. 2A) and AAV virus YL90-treated (FIG. 2B) mdx mice as compared to BL6 (control) mice and untreated mdx (“Mdx4cv uninj”) mice.

FIG. 3 depicts images of muscles from YL90, LW₁, BL6, and Mdx4cv mice immunostained for dystrophin and proteins of the dystrophin-associated-protein complex.

FIG. 4 depicts images of Hematoxylin and Eosin (H&E) stained muscle from LW₁-treated and YL90-treated mice showing a similar muscle histology. Each image is from one independent animal.

FIGS. 5A-5C are graphs depicting muscle force analysis measuring specific twitch force (FIG. 5A), specific force frequency (FIG. 5B), and resistance to stretch-induced damage (percent force drop) (FIG. 5C) in LW₁-treated mdx4cv mice (“LW₁ inj.”), YL90-treated mdx4cv mice (“YL90 inj.”, BL6 (control) mice, and untreated mdx4cv mice (“uninj.”).

FIG. 6 is a schematic illustrating the protein structures of pYL90, pLW₂, pLW₃, pLW₄, pLW₅ , and pLW₆ microdystrophins.

FIGS. 7A-7F are images of muscles immunostained for dystrophin from YL90 (FIG. 7A), LW₂ (FIG. 7B), LW₃ (FIG. 7C), LW₄ (FIG. 7D), LW₅ (FIG. 7E), and LW₆ (FIG. 7F) Mdx4cv mice.

FIGS. 8A-8F are Western blots depicting expression of YL90 (FIG. 8A), LW₂ (FIG. 8B), LW₃ (FIG. 8C), LW₄ (FIG. 8D), LW₅ (FIG. 8E), and LW₆ (FIG. 8F) in Mdx4cv mice.

FIGS. 9A-9F depicts images of H&E stained muscle from YL90 (FIG. 9A), LW₂ (FIG. 9B), LW₃ (FIG. 9C), LW₄ (FIG. 9D), LW₅ (FIG. 9E), and LW₆ (FIG. 9F) in Mdx4cv mice.

FIG. 10 is a schematic illustrating the protein structures of pYL90 (plasmid with full-length microdystrophin) and pLW₃ (plasmid with partial deletion in H4) microdystrophin as compared to BL6 (control) mice and untreated mdx (“Mdx4cv uninj”) mice

FIGS. 11A & 11B are Western blots depicting expression of LW₃-treated (FIG. 11A) and YL90-treated (FIG. 11B) Mdx4cv mice as compared to dystrophin in BL6 mice.

FIGS. 12A-12C are Western blots depicting LW₃ (FIG. 12A) and Vinculin (FIG. 12B) expression and a graph quantifying expression levels of LW₃ and YL90 (FIG. 12C).

FIG. 13 are images of muscles from YL90, LW₃, BL6, and Mdx4cv mice immunostained for dystrophin and proteins of the dystrophin-associated-protein complex.

FIG. 14 depicts images of H&E stained muscle from LW₃-treated and YL90-treated mice showing a similar muscle histology. Each image is from one independent animal.

FIGS. 15A & 15B are graphs depicting muscle force analysis measuring specific twitch force (FIG. 15A) and resistance to stretch-induced damage (percent force drop) (FIG. 15B) in LW₃-treated mdx4cv mice (“LW₃”), YL90-treated mdx4cv mice (“YL90”), BL6 (control) mice, and untreated mdx4cv mice.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The present disclosure is directed to compositions and methods for treating dystrophinopathies.

In one aspect, the present disclosure is directed to a nucleic acid encoding a modified dystrophin comprising a hinge region modification to at least one of hinge 1 (H1) region, hinge 4 (H4) region, and combinations thereof.

The hinge region modification to the hinge 1 (H1) region can be chosen (or selected) from a partial deletion of the H1 region and a complete deletion of the H1 region of the dystrophin protein. A particularly suitable deletion of the H1 region can be a complete deletion of the H1 region of the dystrophin protein. The complete or partial deletion of the H1 region can include amino acid residues from 253 to 327 using the human dystrophin amino acid sequence (GI: M18533.1) as a reference sequence (SEQ ID NO:1). The complete or partial deletion of the H1 region can include amino acid residues from 253 to 329 using the mouse dystrophin amino acid sequence (GI: M68859.1) as a reference sequence (SEQ ID NO:2). SEQ ID NO:3 provides the amino acid sequence of human dystrophin H1 region. SEQ ID NO:4 provides the amino acid sequence of mouse dystrophin H1 region.

The hinge region modification to the hinge 4 (H4) region of dystrophin can be a partial deletion of the H4 region. The partial deletion of the H4 region can include deletion of amino acid residues spanning amino acid residue 3014 to amino acid residue 3112 using the human dystrophin amino acid sequence (GI: M18533.1) as a reference sequence (SEQ ID NO:1). In one embodiment, the partial deletion of the H4 region can include deletion of amino acid residues spanning amino acid residue 3014 to amino acid residue 3055 using the human dystrophin amino acid sequence (GI: M18533.1) as a reference sequence (SEQ ID NO:1). In one embodiment, the partial deletion of the H4 region can include amino acid residues from 3041 to 3112 using the human dystrophin amino acid sequence (GI: M18533.1) as a reference sequence (SEQ ID NO:1). The partial deletion of the H4 region can include amino acid residues from 3034 to 3105 using the mouse dystrophin amino acid sequence (GI: M68859.1) as a reference sequence (SEQ ID NO:2). SEQ ID NO:5 provides the amino acid sequence of human dystrophin H4 region. SEQ ID NO:6 provides the amino acid sequence of mouse dystrophin H4 region.

Suitable modified dystrophin can have a nucleotide sequence of SEQ ID NO:7, SEQ ID NO: 9, SEQ ID NO:11, SEQ ID NO:13, SEQ ID NO:15, SEQ ID NO:17. Suitable modified dystrophin can have a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO:7, SEQ ID NO:9, SEQ ID NO:11, SEQ ID NO:13, SEQ ID NO:15, SEQ ID NO:17.

Percent identity of two sequences can be determined by aligning the sequences for optimal comparison. For example, gaps can be introduced in the sequence of a first nucleic acid sequence for optimal alignment with the second nucleic acid sequence. The same can be done for optimal alignment of amino acid sequences. The nucleotides or amino acid residues at corresponding positions are then compared. When a position in the first sequence is occupied by the same nucleotide or amino acid as at the corresponding position in the second sequence, the nucleic acids or amino acids are identical at that position. The percent identity between the two sequences is a function of the number of identical nucleotides or amino acids shared by the sequences. Hence, percent identity=[number of identical nucleotides/total number of overlapping positions]×100 or percent identity=[number of identical amino acids/total number of overlapping positions]×100. The percentage of sequence identity can be calculated according to this formula by comparing two optimally aligned sequences being compared, determining the number of positions at which the identical nucleic acid or amino acid occurs in both sequences to yield the number of matched positions (the “number of identical positions” in the formula above), dividing the number of matched positions by the total number of positions being compared (the “total number of overlapping positions” in the formula above), and multiplying the result by 100 to yield the percent sequence identity. In this comparison, the sequences can be the same length or may be different in length. Optimal alignment of sequences for determining a comparison window can be conducted by the local homology algorithm of Smith and Waterman (1981), by the homology alignment algorithm of Needleman and Wunsh (1972), by the search for similarity via the method of Pearson and Lipman (1988), by computerized implementations of these algorithms (GAP, BESTFIT, FASTA and TFASTA in the Wisconsin Genetics Software Package Release 7.0, Genetic Computer Group, 575, Science Drive, Madison, Wis.), or by inspection.

Suitable modified dystrophin proteins can have an amino acid sequence of SEQ ID NO:8, SEQ ID NO: 10, SEQ ID NO:12, SEQ ID NO:14, SEQ ID NO:16, SEQ ID NO:18. Suitable dystrophin proteins can have an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO:8, SEQ ID NO: 10, SEQ ID NO:12, SEQ ID NO:14, SEQ ID NO:16, SEQ ID NO:18.

In one aspect, the present disclosure is directed to a vector comprising a nucleic acid encoding a modified dystrophin comprising a hinge region modification to at least one of hinge 1 (HI) region, hinge 4 (H4) region, and combinations thereof.

Particularly suitable vector constructs are expression vector constructs. Suitable vectors include viral vectors and non-viral vectors. Suitable viral vectors are chosen (or selected) from lentiviral vectors and adeno-associated-virus vectors. Suitable vectors include AAV serotypes such as, for example, adeno-associated-virus serotype-1 (AVV-1), adeno-associated-virus serotype-5 (AVV-5), adeno-associated-virus serotype-6 (AVV-6), adeno-associated-virus serotype-8 (AVV-8), adeno-associated-virus serotype-9 (AVV-9), adeno-associated-virus serotype-rh74 (AVV-rh74), adeno-associated-virus-2i8 (AVV-2i8), adeno-associated-virus-Bl (AVV-B 1) , adeno-associated-virus-CAM130 (AVV-CAM130), adeno-associated-virus-M41 (AVV-M41), adeno-associated-virus MTP (AAV587MTP and AAV588MTP), adeno-associated-virus NP22 (AAV-NP22), adeno-associated-virus NP66 (AAV-NP66), adeno-associated-virus MYO (AAVMYO), adeno-associated-virus tyrosine mutants, and ancestral adeno-associated-virus (ancAVV). Suitable vectors also include plasmid, liposome, exosome, and nanoparticles.

The exact details of the vector construct vary according to the particular host cell that is to be used as well as to the desired characteristics of the expression system, as is well known in the art. For example, promoter sequences compatible with bacterial hosts are typically provided in plasmid vectors containing one or more convenient restriction sites for insertion of a contemplated nucleic acid segment. Suitable promoters and vectors include the Rec 7 promoter that is inducible by exogenously supplied nalidixic acid, JHEX25 (commercially available from Promega, Madison, Wis.) that is inducible by exogenously supplied isopropyl-β-D-thiogalacto-pyranoside (IPTG), tac (a hybrid of the trp and lac promoter/operator) present in plasmid vector pKK223-3 (commercially available from Pharmacia, Piscataway, N.J.) and is also inducible by exogenously supplied IPTG. Other suitable promoters and promoter/operators include the araB, trp, lac, gal, T7, and the like. For production in S. cerevisiae, the nucleic acid encoding a thrombin precursor of the disclosure is placed into operable linkage with a promoter that is operable in S. cerevisiae and which has the desired characteristics (e.g., inducible/derepressible or constitutive), such as GAL1-10, PHOS5, PGK1, GDP1, PMA1, MET3, CUP1, GAP, TPI, MFα1 and MFα2, as well as the hybrid promoters PGK/α2, TPI/α2, GAP/GAL, PGK/GAL, GAP/ADH2, GAP/PHO5, ADH2/PHO5, CYC1/GRE, and PGK/ARE and other promoters known in the art. For a mammalian cell line, the promoter can be a viral promoter/enhancer (e.g., the herpes virus thymidine kinase (TK) promoter or a simian virus promoter (e.g., the SV40 early or late promoter) or the Adenovirus major late promoter, a long terminal repeat (LTR), such as the LTR from cytomegalovirus-(CMV), Rous sarcoma virus (RSV) or mouse mammary tumor virus (MMTV)) or a mammalian promoter, suitably an inducible promoter such as the metallothionein or glucocorticoid receptor promoters and the like. For muscle, suitable promoters include an endogenous and synthetic heart-specific or muscle-specific promoters (e.g., SPc5-12, muscle creatine kinase (see e.g., Wang et al., Gene Ther. 2008 Nov;15(22):1489-99, which is incorporated by reference), desmin, MYOD1, and MYLK2.

Constructs can include additional nucleic acids appropriate for the intended host cell. For example, expression constructs for use in higher eukaryotic cell lines (e.g., vertebrate and insect cell lines) include a polyadenylation site and can include an intron (including signals for processing the intron), as the presence of an intron appears to increase mRNA export from the nucleus in many systems. Additionally, a secretion signal sequence operable in the host cell can be included as part of the construct. Other suitable secretion signal sequences can be obtained from human serum albumin, human prothrombin, human tissue plasminogen activator, and preproinsulin. Expression constructs may also contain other commonly used regulator elements such as microRNA target site to reducing expression in non-targeted tissues/cells. Expression constructs may also contain elements that are used to express two transgenes such as IRES and 2A. Where the expression construct is intended for use in a prokaryotic cell, the expression construct can include a signal sequence that directs transport of the synthesized polypeptide into the periplasmic space or expression can be directed intracellularly. Constructs can also selectable markers for selecting host cells that contain the construct. Selectable markers are well known in the art. Marker genes contained in the expression vector for a microorganism can be, for example, an ampicillin resistance gene, tetracycline resistance gene for E. coli as a host; Leu2 gene for yeast as a host, and the like. Marker genes contained in the expression vector for an animal cell can be, for example, aminoglycoside 3′phosphotransferase (neo) gene, dihydrofolate reductase (dhfr) gene, glutamine synthetase (GS) gene, and the like.

Suitable modified dystrophins include modifications to the H1 region, the H4 region, and combinations thereof, as described herein.

In another aspect, the present disclosure is directed to a host cell comprising a vector, wherein the vector comprises a nucleic acid encoding a modified dystrophin.

Suitable host cells include, for example, eukaryotic host cells and prokaryotic host cells. Suitable eukaryotic cells include muscle cells and cardiac cells including primary cultured muscle cells, primary cultured cardiac cells, immortalized muscle cells, and immortalized cardiac cells. Suitable eukaryotic cells include insect cells such as Sf9, and mammalian cell lines such as CHO, COS, 293, 293-EBNA, BHK, HeLa, NIH/3T3, and the like. Exemplary yeast host cells include Saccharomyces cerevisiae, Pichia pastoris, Hansenula polymorpha, Kluyveromyces lactis, Schwanniomyces occidentis, Schizosaccharomyces pombe, Arxula adeninivorans, Candida boidinii, Hansenula polymorpha, and Yarrowia lipolytica. Suitable prokaryotic cells are bacteria cells including, for example, E. coli cells such as, for example, BL21 (DE3), XL-1, TB1, JM103, BLR, pUC8, pUC9, pBR329, pPL, SURE, SUREII, DH5a, Stb12, Stb13, Top10 and pKK223-3 cells and Salmonella such as, for examples, S. typhi, S. typhimurium and S. typhimurium-E. coli hybrids.

Modified dystrophin polypeptides of the present disclosure can be prepared by incorporating a nucleic acid encoding the polypeptides into an expression vector, transforming suitable microorganism or animal cells with the resulting expression vector, and culturing the transformed microorganism or animal cells to produce the polypeptides encoded by the nucleic acid. For production of the polypeptides encoded by the nucleic acid, a peptide synthesizer can also be used.

Suitable vectors include viral vectors and non-viral vectors. Suitable vectors include AAV serotypes such as, for example, adeno-associated-virus serotype-1 (AVV-1), adeno-associated-virus serotype-5 (AVV-5), adeno-associated-virus serotype-6 (AVV-6), adeno-associated-virus serotype-8 (AVV-8), adeno-associated-virus serotype-9 (AVV-9), adeno-associated-virus serotype-rh74 (AVV-rh74), adeno-associated-virus-2i8 (AVV-2i8), adeno-associated-virus-Bl (AVV-B1), adeno-associated-virus-CAM130 (AVV-CAM130), adeno-associated-virus-M41 (AVV-M41), adeno-associated-virus MTP (AAV587MTP and AAV588MTP), adeno-associated-virus NP22 (AAV-NP22), adeno-associated-virus NP66 (AAV-NP66), adeno-associated-virus MYO (AAVMYO), adeno-associated-virus tyrosine mutants, and ancestral adeno-associated-virus (ancAVV). Suitable vectors also include plasmid, liposome, exosome, and nanoparticles.

Suitable modified dystrophins include modifications to the H1 region, the H4 region, and combinations thereof, as described herein.

Nucleic acids encoding secretion signal sequences for secretion in microorganism or animal cell expression cultures can be included in the nucleic acid encoding the modified dystrophin. The modified dystrophin of the present disclosure can be expressed and secreted into a culture medium. Suitable signal sequences include, for example, pel B signal; a factor signal; immunoglobulin signal SG-1, C25 signal, and the like. A particularly suitable secretion signal sequence is a factor V secretion peptide.

Sequences for tags can be included in a nucleic acid encoding the modified dystrophin of the present disclosure. Suitable tags can be purification tags and labels. Suitable purification tags can be histidine, HPC4, GST, C-tag, c-myc, T7, Glu-Glu, FLAG, HA, MBP, CBP, intein-CBD, Streptavidin/Biotin-based tag, SUMO-tag, and HaloTAG tags.

Sequences encoding restriction sites can be included in a nucleic acid encoding the modified dystrophin of the present disclosure.

A variety of animal cells can be used as a host cell as described herein. A host cell can be transformed by any known methods including, for example, a calcium phosphate method, a DEAE dextran method, precipitation with e.g. lipid-based transfection reagents (e.g. lipofectin), fusion of protoplast with polyethylene glycol, electroporation, biolistic, and the like. A particularly suitable method for transfection is LIPOFECTAMINE® 3000.

In one aspect, the present disclosure is directed to a method for treating a dystrophinopathy. The method includes administering a modified dystrophin having a modified hinge region. Suitable modified hinge regions include modifications to the H1 region, the H4 region, and combinations thereof, as described herein.

Suitable carriers include water, saline, isotonic saline, phosphate buffered saline, Ringer's lactate, and the like.

Formulations for delivering the modified dystrophin can also include other components such as surfactants, preservatives, and excipients. Surfactants can reduce or prevent surface-induced aggregation of the dystrophin microgenes. Suitable surfactants fatty acid esters and alcohols, and polyoxyethylene sorbitol fatty acid esters. Amounts will generally range from about 0.001 and about 4% by weight of the formulation. Pharmaceutically acceptable preservatives include, for example, phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesin (3p-chlorphenoxypropane-1,2-diol) and mixtures thereof. The preservative can be present in concentrations ranging from about 0.1 mg/ml to about 20 mg/ml, including from about 0.1 mg/ml to about 10 mg/ml. The use of a preservative in pharmaceutical compositions is well-known to those skilled in the art. For convenience reference is made to Remington: The Science and Practice of Pharmacy, 19th edition, 1995. Formulations can include suitable buffers such as sodium acetate, glycylglycine, HEPES (4-(2-hydroxyethyl)-1-piperazineethanesulfonic acid) and sodium phosphate. Excipients include components for tonicity adjustment, antioxidants, and stabilizers as commonly used in the preparation of pharmaceutical formulations. Other inactive ingredients include, for example, L-histidine, L-histidine monohydrochloride monohydrate, sorbitol, polysorbate 80, sodium citrate, sodium chloride, and EDTA disodium.

In one embodiment, the carrier is a pharmaceutically acceptable carrier. As understood by those skilled in the art, pharmaceutically acceptable carriers, and, optionally, other therapeutic and/or prophylactic ingredients must be “acceptable” in the sense of being compatible with the other ingredients of the formulation and not be harmful to the recipient thereof. Suitable pharmaceutically acceptable carrier solutions include water, saline, isotonic saline, phosphate buffered saline, Ringer's lactate, and the like. The compositions of the present disclosure can be administered to animals, preferably to mammals, and in particular to humans as therapeutics per se, as mixtures with one another or in the form of pharmaceutical preparations, and which as active constituent contains an effective dose of the active agent, in addition to customary pharmaceutically innocuous excipients and additives.

Formulations for parenteral administration (e.g. by injection, for example bolus injection or continuous infusion) can be presented in unit dose form in ampoules, pre-filled syringes, small volume infusion or in multi-dose containers with and without an added preservative. The formulations can take such forms as suspensions, solutions, or emulsions in oily or aqueous vehicles, and may contain formulation agents such as suspending, stabilizing and/or dispersing agents.

Suitable methods for administration of formulations of the present disclosure are by parenteral (e.g., intravenous (IV), intramuscular (IM), subcutaneous (SC), or intraperitoneal (IP)) routes and the formulations administered ordinarily include effective amounts of product in combination with acceptable diluents, carriers and/or adjuvants. Standard diluents such as human serum albumin are contemplated for pharmaceutical compositions of the disclosure, as are standard carriers as described herein.

As used herein, an “effective amount”, a “therapeutically effective amount”, a “prophylactically effective amount” and a “diagnostically effective amount” is the amount of the modified dystrophin of the present disclosure needed to elicit the desired biological response following administration. The amount of the modified dystrophin will depend on the form the modified dystrophin is in such as whether it is administered as a nucleic acid encoding the modified dystrophin (including being packaged in an expression construct and/or vector) or as a modified dystrophin protein.

Effective dosages are expected to vary substantially depending upon the modified dystrophin used and the specific disease, disorder, or condition treated. Dosages can range from about 10¹⁰ vector genomes per kilogram to about 10¹⁴ vector genomes per kilogram. Suitable dosage for use in the methods of the present disclosure will depend upon a number of factors including, for example, age and weight of an individual, the specific dystrophinopathy, severity of the dystrophinopathy, nature of a composition, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

Off-target effects can be minimized using muscle-specific regulatory cassettes such as those derived from the MCK (such as miniMCK, CKS, CK6, CK7, CK8, CK8e, CK9 and MHCK7) gene, myoglobin gene and desmin genes, cardiac promoters (such as cTN1, NCX1, MLC-2v, alpha-1c, mini-alphaMHC), Pitx3, skeletal muscle alpha-actin, and synthetic promoters (such as SPc5-12, SK-CRM1, SK-CRM2, SK-CRM3, SK-CRM4, SK-CRMS, SK-CRM6, SK-CRM7, SK-CRM-Des, SK-CRM-SPc5-12, SK448, and SP1-28). Gene expression can be enhanced using stronger regulatory cassettes and using codon-optimized, functionally enhanced cDNAs.

Formulations of the present disclosure can be administered to subjects in need thereof. As used herein, “a subject” (also interchangeably referred to as “an individual” and “a patient”) refers to animals including humans and non-human animals. Accordingly, the compositions and methods disclosed herein can be used for human and veterinarian applications, particularly human and veterinarian medical applications. Suitable subjects include warm-blooded mammalian hosts, including humans, companion animals (e.g., dogs, cats), cows, horses, mice, rats, rabbits, primates, and pigs, preferably a human patient.

In another aspect, the present disclosure is directed to a method for treating a dystrophinopathy in a subject in need thereof. The method includes administering a modified dystrophin to the subject in need thereof.

As used herein, “a subject in need thereof” (also used interchangeably herein with “a patient in need thereof”) refers to a subject susceptible to or at risk of a specified disease, disorder, or condition. The methods disclosed herein can be used with a subset of subjects who are susceptible to or at elevated risk for dystrophinopathies. Because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions. In one embodiment, the subject has or is suspected of having a dystrophinopathy. In one embodiment, the subject is a carrier of a dystrophinopathy. As used herein, a “carrier” (or “hereditary carrier”) of a dystrophinopathy refers to a subject that has inherited a recessive allele for a genetic trait or mutation known or believed to cause a dystrophinopathy. A carrier may not show any symptoms of the dystrophinopathy or may show mild symptoms such as muscle weakness, cramps, cardiomyopathy, and combinations thereof.

Dystrophinopathies include diseases caused by or resulting from a mutation in the dystrophin gene. Suitable dystrophinopathies include Duchenne muscular dystrophy (DMD), Becker muscular dystrophy (BMD), X-linked dialated cardiomyopathy (XLDC) and their carriers.

Suitable methods for administration of formulations of the present disclosure are by parenteral (e.g., IV, IM, SC, or IP) routes as described herein.

In another aspect, the present disclosure is directed to a method for treating dystrophinopathy in a subject in need thereof. The method includes: administering a modified dystrophin protein, the modified dystrophin protein comprising a hinge region modification to at least one of hinge 1 (H1) region, hinge 4 (H4) region, and combinations thereof.

Dystrophinopathies include diseases caused by or resulting from a mutation in the dystrophin gene. Suitable dystrophinopathies include Duchenne muscular dystrophy (DMD), Becker muscular dystrophy (BMD), X-linked dialated cardiomyopathy (XLDC) and their carriers, as described herein.

Suitable methods for administration of formulations of the present disclosure are by parenteral (e.g., IV, IM, SC, or IP) routes as described herein.

The modified dystrophin protein can be delivered as a composition including a carrier as described herein. Particularly suitable carriers can be polymers. Particularly suitable polymers can be poloxamers.

Suitable dosage of modified dystrophin protein for use in the methods of the present disclosure will depend upon a number of factors including, for example, age and weight of an individual, the specific dystrophinopathy, severity of the dystrophinopathy, nature of a composition, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

While the methods of the present disclosure may be used to isolate and identify some interactions having a long interaction half-life, advantageously, the method also allows for the isolation of weakly interacting molecules.

The disclosure will be more fully understood upon consideration of the following non-limiting Examples.

### EXAMPLES

### Materials and Methods

Construction of LW1 and LW3 plasmids and intramuscular injections in mdx4cv mice. The original plasmids were generated using YL90 as the initial backbone and using a PCR based deletion strategy. YL90 was prepared as described in Lai, Thomas et al. (2009 J Clin Invest 119:624-35). YL90 with complete deletion of hinge 1 region (LW₁) and YL90 with partial deletion of H4 region (LW₃) were prepared. The plasmids were confirmed by sequencing and diagnostic digestion. Adeno-associated-virus serotype-9 (AAV-9) carrying LW₁, LW₃ or YL90 were generated using these plasmids for intramuscular injections in mice. All animal experiments were approved by the institutional animal care and use committee and were in accordance with NIH guidelines. The tibialis anterior (TA) muscle of the mdx4cv mice were injected at a dose of 1E12vg/muscle in 3-m-old mice. The contractile properties of the TA muscle were evaluated at 3-months post-injection.

TA muscle function evaluation. The TA muscle force was measured in situ according to our published protocol (Hakim, Li et al. 2011 Methods Mol Biol 709: 75-89; Hakim, Wasala et al. 2013 J Vis Exp: e50183). In particular, mice were anesthetized via intra-peritoneal injection of a cocktail containing 25 mg/ml ketamine, 2.5 mg/ml xylazine and 0.5 mg/ml acepromazine at 2.5 μl/g body weight. The TA muscle and the sciatic nerve were exposed. The mouse was transferred to a custom-designed thermo-controlled platform of the footplate apparatus (Hakim, Li et al. 2011 Methods Mol Biol 709: 75-89; Hakim, Wasala et al. 2013 J Vis Exp: e50183). After 5 minutes of equilibration, the sciatic nerve was stimulated at the frequency of 1 Hz (20V, 1,000 mA) to elicit twitch muscle contraction using a custom-made 25G platinum electrode at 2.0-6.0 g resting tensions. The muscle length (Lₘ) of the TA muscle was measured with an electronic digital caliper (Fisher Scientific, Waltham, Mass., USA) at the resting tension that generated the maximal twitch force. This length was defined as the optimal muscle length (L₀). The twitch force was measured at 1 Hz frequency followed by the force frequency assay at 50, 100, 150 and 200 Hz with 1 min resting between each contraction. Specific muscle force was determined by dividing the maximum isometric tetanic force with the muscle cross sectional area (CSA). The CSA was calculated according to the following equation, CSA=(muscle mass, in gram)/[(optimal fiber length, in cm)×(muscle density, in g/cm³)]. A muscle density of 1.06 g/cm³ was used in calculation. Optimal fiber length was calculated as 0.60×L₀. 0.60 represents the ratio of the fiber length to the L₀ of the TA muscle. After tetanic force measurement, the muscle was rested for 5 min and then subjected to ten rounds of eccentric contraction according to previously published protocols (Hakim, Li et al. 2011 Methods Mol Biol 709: 75-89; Hakim, Wasala et al. 2013 J Vis Exp: e50183). Briefly, following a tetanic contraction the TA muscle was stretched by 10%Lo at a rate 0.5L₀/sec. The muscle was allowed to rest 1 min between each eccentric contraction cycle. The percentage of force drop following each round of eccentric contraction was recorded. Muscle twitch and tetanic forces and the eccentric contraction profile were measured with a 305C-LR dual-mode servomotor transducer (Aurora Scientific, Inc.). Data were processed using the Lab View-based DMC and DMA programs (Version 3.12, Aurora Scientific, Inc.).

Morphological studies. General histology was examined by hematoxylin and eosin (HE) staining. Dystrophin expression was evaluated by immunofluorescence staining using the MANEX44A dystrophin antibody specific to exons 44 (1:100; clone 5B2) was obtained from MDA Monoclonal Antibody Resource located at the Wolfson Centre for Inherited Neuromuscular Disease, RJAH Orthopaedic Hospital, Oswestry, UK (www.glennmorris.org.uk/mabs.htm). Slides were viewed at the identical exposure setting using a Nikon E800 fluorescence microscope. Photomicrographs were taken with a Qimage REtiga 1300 camera.

Western blot. TA muscles lysates were prepared as described (Li, Long, et al. 2009 Hum Mol Genet 18: 1209-20). Briefly, the tissues were snap frozen in liquid nitrogen. The frozen tissue samples were ground to fine powder in liquid nitrogen followed by homogenization in a buffer containing 10% SDS, 5 mM EDTA, 62.5 mM Tris-HCl at pH6.8 and the protease inhibitor cocktail (Roche, Indianapolis, Ind.). The crude lysate was heated at 95° C. for 3 min, chilled on ice for 2 min and then centrifuged at 14,000 rpm for 3 min Supernatant was collected as the whole muscle lysate. Protein concentration was measured using the DC protein assay kit (Bio-Rad, Hercules, Calif.) and 100 μg of protein was used to load per lane for the western blot. Dystrophin was detected with MANEX1011D dystrophin antibody specific to exons 10-11 (1:100; clone 7G5, IgG1), was obtained from MDA Monoclonal Antibody Resource located at the Wolfson Centre for Inherited Neuromuscular Disease, RJAH Orthopaedic Hospital, Oswestry, UK. Western blot quantification was performed using the LI-COR Image Studio Version 5.0.21 software. The relative intensity of the respective protein band was normalized to the corresponding loading control in the same blot.

As used in the Examples, pXX refers to the plasmid containing the microdystrophin. Thus, pYL90 refers to a plasmid with the full-length microdystrophin; pLW₁ refers to a plasmid with the ΔH₁ microdystrophin (complete deletion of H1); pLW₂ refers to a plasmid with the complete deletion of amino acid residues 3041 to 3112 of H4; pLW₃ refers to a plasmid with the partial deletion of amino acid residues 3041 to 3055 of H4; pLW₄ refers to a plasmid with the partial deletion of amino acid residues 3093 to 3112 of H4; pLW₅ refers to a plasmid with the deletion of amino acid residues 3041 to 3055 and 3093 to 3112 of H4 and WW domain of Dp427 was retained; and pLW₆ refers to a plasmid with the deletion of amino acid residues 3041 to 3075 and 3093 to 3112 of H4, the partial WW domain from amino acid residues 3076 to 3092 present in Dp71 was retained. As used in the Examples, XX refers to the AAV virus containing the microdystrophin. Thus, YL90 refers to AAV virus with the full-length microdystrophin; LW₁ refers to AAV virus with the AH₁ microdystrophin (complete deletion of HI); LW₂ refers to AAV virus with the complete deletion of amino acid residues 3041 to 3112 of H4; LW₃ refers to AAV virus with the partial deletion of amino acid residues 3041 to 3055 of H4; LW₄ refers to AAV virus with the partial deletion of amino acid residues 3093 to 3112 of H4; LW₅ refers to AAV virus with the deletion of amino acid residues 3041 to 3055 and 3093 to 3112 of H4 and WW domain of Dp427 was retained; and LW₆ refers to AAV virus with the deletion of amino acid residues 3041 to 3075 and 3093 to 3112 of H4, the partial WW domain from amino acid residues 3076 to 3092 present in Dp71 was retained.

### Example 1

In this Example, the effect of modifications to hinge 1 region in microdystrophin was determined

In particular, a full-length microdystrophin (pYL90) was used as the parental microgene. Hinge 1 of pYL90 was completely deleted to render pLW₁ microdystrophin without H1 (plasmid with a complete deletion of the H1 in the microdystrophin; AH₁ microdystrophin). FIG. 1 illustrates the protein structure of pYL90 and pLW₁.

Both microdystrophins were packaged into AAV-9 adeno virus vector under the control of the CMV promoter to produce AAV virus YL90 and AAV virus pLW₁ and injected into the tibialis anterior muscles of 3 month old male mdx4cv mice at a dosage of 1 el2vg/muscle. Muscle force analysis, immunological staining, and histological staining were done at 3 months post-injection.

As shown in FIGS. 2A and 2B, expression of LW₁ (referring to AAV virus with the pLW₁ plasmid) (FIG. 2A) was similar to expression of YL90 (referring to AAV virus with the pYL90 plasmid) (FIG. 2B).

Muscle tissue from BL6 control mice, Mdx4cv mice (dystrophin deficient), Mdx4cv mice injected with LW₁, and Mdx4cv mice injected with YL90 were immunostained with antibodies against dystrophin, dystrovrevin, α-syntrophin, β-sarcoglycan, and β-dystroglycan to visualize the dystrophin associated protein complex. As depicted in FIG. 3, LW₁ and YL90 showed a similar staining pattern. Both proteins also restored the DGC (FIG. 3). LW₁-treated and YL90-treated mice showed a similar muscle histology (FIG. 4). LW₁-treated (“LW₁ inj”) and YL90-treated (“YL90 inj”) mice had specific twitch force (FIG. SA) and specific force frequency (FIG. 5B) similar to BL6 mice. LW₁-treated (“LW₁ inj”) and YL90-treated (“YL90 inj”) mice had similar resistance to stretch-induced damage (percent force drop) (FIG. SC). These results demonstrate that LW₁ and YL90 were equally effective in restoring muscle force in dystrophin-deficient mice.

### Example 2

In this Example, the effect of modifications to hinge 4 region in microdystrophin was determined.

As in Example 1 above, full-length microdystrophin (pYL90) was used as the parental microgene. FIG. 6 illustrates the protein structure of pYL90 (plasmid with the full-length microdystrophin), pLW₂ (plasmid with the complete deletion of amino acid residues 3041 to 3112 of H4) pLW₃ (plasmid with the partial deletion of amino acid residues 3041 to 3055 of H4), pLW₄ (plasmid with the partial deletion of amino acid residues 3093 to 3112 of H4), pLW₅ (plasmid with the deletion of amino acid residues 3041 to 3055 and 3093 to 3112 of H4; WW domain of Dp427 was retained), pLW₆ (plasmid with the deletion of amino acid residues 3041 to 3075 and 3093 to 3112 of H4; the partial WW domain from amino acid residues 3076 to 3092 present in Dp71 was retained).

As depicted in FIG. 7, immunostaining revealed normal sarcolemmal localization of LW₃ but not LW₂, LW₄, LW₅ and LW₆. LW₂, LW₄, LW₅ and LW₆ resulted in different levels of cytosolic aggregate formation. FIGS. 8A-8B depict Western blot evaluation of YL90 (FIG. 8A), LW₂ (FIG. 8B), LW₃ (FIG. 8C), LW₄ (FIG. 8D), LW₅ (FIG. 8E), and LW₆ (FIG. 8F). As shown in FIG. 8D, LW₄ showed reduced expression. As shown in FIG. 8F, no LW₆ microdystrophin was detected. YL90 (FIG. 8A), LW₂ (FIG. 8B), LW₃ (FIG. 8C), and LW₅ (FIG. 8E) showed similar levels of expression.

As shown in FIGS. 9A-9F (H&E staining of muscle), only LW₃ (FIG. 9C) showed similar histology to YL90 (FIG. 9A). Muscle histology in H&E stained muscle appeared worse in LW₂ (FIG. 9B), LW₄ (FIG. 9D), LW₅ (FIG. 9E) and LW₆ (FIG. 9F).

LW₃ and YL90 microdystrophins (FIG. 10) were packaged into AAV-9 and injected in the tibialis anterior muscle of 3 month old male mdx4cv mice at 1 el2vg/muscle. Muscle force analysis, immunological and histological staining was carried out at 3 months post-injection.

As shown in FIGS. 11A and 11B, LW₃ expression (FIG. 11A) appeared higher than YL90 expression (FIG. 11B). FIGS. 11A and 11B also show dystrophin in BL6 mice and no dystrophin in Mdx4cv mice that were not injected (“Mdx4cv uninj” lanes) with either LW₃ or YL90. FIG. 12A is a Western blot with LW₃ or YL90 samples on the same blot and shows that the expression of LW₃ was slightly lower than the expression of YL90. FIG. 12B is a Western blot analysis detecting vinculin to show a loading control. FIG. 12C shows that the expression of LW₃ was slightly lower than the expression of YL90, but not statistically significant.

As shown in FIG. 13, LW₃ and YL90 showed similar staining patterns in immunostained muscle. Additionally, both LW₃ and YL90 restored DGC. LW₃-treated and YL90-treated mice showed similar muscle histology (FIG. 14). As depicted in FIG. 15, LW₃-treatment resulted in higher muscle force (specific twitch and tetanic force than YL90-treatment.

These results demonstrated that the methods and compositions of the present disclosure can be used to treat dystrophinopathies. The microdystrophins and methods of the present disclosure provide alternative microdystrophins for treating dystrophinopathies.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A nucleic acid encoding a modified dystrophin comprising a hinge region modification to at least one of a modification of hinge 1 (H1) region, a modification of hinge 4 (H4) region, and combinations thereof.

2. The nucleic acid of claim 1, wherein the modification of hinge 1 (H1) region is chosen from a complete deletion of hinge 1 (H1) region and a partial deletion of hinge 1 (H1) region.

3. The nucleic acid of claim 1, wherein the modification of hinge 4 region is a partial deletion of hinge 4 (H4) region.

4. A vector comprising a nucleic acid encoding a modified dystrophin comprising a hinge region modification to at least one of a modification of hinge 1 (H1) region, a modification of hinge 4 (H4) region, and combinations thereof.

5. The vector of claim 4, wherein the modification of H1 region is chosen from a complete deletion of hinge 1 (H1) region and a partial deletion of hinge 1 (H1) region.

6. The vector of claim 4, wherein the modification of hinge 4 (H4) region is a partial deletion of hinge 4 (H4) region.

7. The vector of claim 4, wherein the vector is chosen from a viral vector and a non-viral vector.

8. The vector of claim 7, wherein the viral vector is chosen from a lentiviral vector and an adeno-associated-virus vector.

9. The vector of claim 8, wherein the adeno-associated-virus vector is chosen from adeno-associated-virus serotype-1 (AVV-1), adeno-associated-virus serotype-5 (AVV-5), adeno-associated-virus serotype-6 (AVV-6), adeno-associated-virus serotype-8 (AVV-8), adeno-associated-virus serotype-9 (AVV-9), adeno-associated-virus serotype-rh74 (AVV-rh74), adeno-associated-virus-2i8 (AVV-2i8), adeno-associated-virus-Bl (AVV-B1), adeno-associated-virus-CAM130 (AVV-CAM130), adeno-associated-virus-M41 (AVV-M41), adeno-associated-virus MTP (AAV587MTP and AAV588MTP), adeno-associated-virus NP22 (AAV-NP22), adeno-associated-virus NP66 (AAV-NP66), adeno-associated-virus MYO (AAVMYO), adeno-associated-virus tyrosine mutants, and ancestral adeno-associated-virus (ancAVV).

10. The vector of claim 4, further comprising a tissue-specific promoter.

11. The vector of claim 10, wherein the tissue-specific promoter is chosen from a muscle-specific promoter and a heart-specific promoter.

12. A method for treating dystrophinopathy in a subject in need thereof, the method comprising:
administering vector to the subject in need thereof, wherein the vector comprises a nucleic acid encoding a modified dystrophin, the modified dystrophin comprising a hinge region modification to at least one of hinge 1 (H1) region, hinge 4 (H4) region, and combinations thereof.

13. The method of claim 12, wherein the hinge region modification is chosen from a partial deletion of hinge 1 (H1) region and a complete deletion of hinge 1 (H1) region.

14. The method of claim 12, wherein the hinge region modification is a partial deletion of hinge 4 (H4) region.

15. The method of claim 12, wherein the nucleic acid is packaged in a vector.

16. The method of claim 15, wherein the vector is chosen from a lentiviral vector and an adeno-associated-virus vector.

17. The method of claim 16, wherein the adeno-associated-virus vector is chosen from adeno-associated-virus serotype-1 (AVV-1), adeno-associated-virus serotype-5 (AVV-5), adeno-associated-virus serotype-6 (AVV-6), adeno-associated-virus serotype-8 (AVV-8), adeno-associated-virus serotype-9 (AVV-9), adeno-associated-virus serotype-rh74 (AVV-rh74), adeno-associated-virus-2i8 (AVV-2i8), adeno-associated-virus-B1 (AVV-B1), adeno-associated-virus-CAM130 (AVV-CAM130), adeno-associated-virus-M41 (AVV-M41), adeno-associated-virus MTP (AAV587MTP and AAV588MTP), adeno-associated-virus NP22 (AAV-NP22), adeno-associated-virus NP66 (AAV-NP66), adeno-associated-virus MYO (AAVMYO), adeno-associated-virus tyrosine mutants, and ancestral adeno-associated-virus (ancAVV).

18. A method for treating dystrophinopathy in a subject in need thereof, the method comprising: administering a modified dystrophin protein, the modified dystrophin protein comprising a hinge region modification to at least one of hinge 1 (H1) region, hinge 4 (H4) region, and combinations thereof.

19. The method of claim 18, wherein the dystrophinopathy is chosen from Duchenne muscular dystrophy (DMD), Becker muscular dystrophy (BMD), and X-linked dilated cardiomyopathy (XLDC).

20. The method of claim 18, wherein the subject is a carrier of a dystrophinopathy chosen from Duchenne muscular dystrophy (DMD), Becker muscular dystrophy (BMD), and X-linked dilated cardiomyopathy (XLDC).


SEQUENCE LISTING (18 sequences)

SEQ ID NO: 1 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 2 - protein, 3678 aa, Mus musculus
MLWWEEVEDCYEREDVQKKTFTKWINAQFSKFGKQHIDNLFSDLQDGKRLLDLLEGLTGQKLPKEKGSTRVHALNNVNKALRVLQKNNVDLVNIGSTDIVDGNHKLTLGLIWNIILHWQVKNVMKTIMAGLQQTNSEKILLSWVRQSTRNYPQVNVINFTSSWSDGLALNALIHSHRPDLFDWNSVVSQHSATQRLEHAFNIAKCQLGIEKLLDPEDVATTYPDKKSILMYITSLFQVLPQQVSIEAIQEVEMLPRTSSKVTREEHFQLHHQMHYSQQITVSLAQGYEQTSSSPKPRFKSYAFTQAAYVATSDSTQSPYPSQHLEAPRDKSLDSSLMETEVNLDSYQTALEEVLSWLLSAEDTLRAQGEISNDVEEVKEQFHAHEGFMMDLTSHQGLVGNVLQLGSQLVGKGKLSEDEEAEVQEQMNLLNSRWECLRVASMEKQSKLHKVLMDLQNQKLKELDDWLTKTEERTKKMEEEPFGPDLEDLKCQVQQHKVLQEDLEQEQVRVNSLTHMVVVVDESSGDHATAALEEQLKVLGDRWANICRWTEDRWIVLQDILLKWQHFTEEQCLFSTWLSEKEDAMKNIQTSGFKDQNEMMSSLHKISTLKIDLEKKKPTMEKLSSLNQDLLSALKNKSVTQKMEIWMENFAQRWDNLTQKLEKSSAQISQAVTTTQPSLTQTTVMETVTMVTTREQIMVKHAQEELPPPPPQKKRQITVDSELRKRLDVDITELHSWITRSEAVLQSSEFAVYRKEGNISDLQEKVNAIAREKAEKFRKLQDASRSAQALVEQMANEGVNAESIRQASEQLNSRWTEFCQLLSERVNWLEYQTNIITFYNQLQQLEQMTTTAENLLKTQSTTLSEPTAIKSQLKICKDEVNRLSALQPQIEQLKIQSLQLKEKGQGPMFLDADFVAFTNHFNHIFDGVRAKEKELQTIFDTLPPMRYQETMSSIRTWIQQSESKLSVPYLSVTEYEIMEERLGKLQALQSSLKEQQNGFNYLSDTVKEMAKKAPSEICQKYLSEFEEIEGHWKKLSSQLVESCQKLEEHMNKLRKFQNHIKTLQKWMAEVDVFLKEEWPALGDAEILKKQLKQCRLLVGDIQTIQPSLNSVNEGGQKIKSEAELEFASRLETELRELNTQWDHICRQVYTRKEALKAGLDKTVSLQKDLSEMHEWMTQAEEEYLERDFEYKTPDELQTAVEEMKRAKEEALQKETKVKLLTETVNSVIAHAPPSAQEALKKELETLTTNYQWLCTRLNGKCKTLEEVWACWHELLSYLEKANKWLNEVELKLKTMENVPAGPEEITEVLESLENLMHHSEENPNQIRLLAQTLTDGGVMDELINEELETFNSRWRELHEEAVRKQKLLEQSIQSAQEIEKSLHLIQESLEFIDKQLAAYITDKVDAAQMPQEAQKIQSDLTSHEISLEEMKKHNQGKDANQRVLSQIDVAQKKLQDVSMKFRLFQKPANFEQRLEESKMILDEVKMHLPALETKSVEQEVIQSQLSHCVNLYKSLSEVKSEVEMVIKTGRQIVQKKQTENPKELDERVTALKLHYNELGAKVTERKQQLEKCLKLSRKMRKEMNVLTEWLAATDTELTKRSAVEGMPSNLDSEVAWGKATQKEIEKQKAHLKSVTELGESLKMVLGKKETLVEDKLSLLNSNWIAVTSRVEEWLNLLLEYQKHMETFDQNIEQITKWIIHADELLDESEKKKPQQKEDILKRLKAEMNDMRPKVDSTRDQAAKLMANRGDHCRKVVEPQISELNRRFAAISHRIKTGKASIPLKELEQFNSDIQKLLEPLEAEIQQGVNLKEEDFNKDMSEDNEGTVNELLQRGDNLQQRITDERKREEIKIKQQLLQTKHNALKDLRSQRRKKALEISHQWYQYKRQADDLLKCLDEIEKKLASLPEPRDERKLKEIDRELQKKKEELNAVRRQAEGLSENGAAMAVEPTQIQLSKRWRQIESNFAQFRRLNFAQIHTLHEETMVVTTEDMPLDVSYVPSTYLTEISHILQALSEVDHLLNTPELCAKDFEDLFKQEESLKNIKDNLQQISGRIDIIHKKKTAALQSATSMEKVKVQEAVAQMDFQGEKLHRMYKERQGRFDRSVEKWRHFHYDMKVFNQWLNEVEQFFKKTQNPENWEHAKYKWYLKELQDGIGQRQAVVRTLNATGEEIIQQSSKTDVNILQEKLGSLSLRWHDICKELAERRKRIEEQKNVLSEFQRDLNEFVLWLEEADNIAITPLGDEQQLKEQLEQVKLLAEELPLRQGILKQLNETGGAVLVSAPIRPEEQDKLEKKLKQTNLQWIKVSRALPEKQGELEVHLKDFRQLEEQLDHLLLWLSPIRNQLEIYNQPSQAGPFDIKEIEVTVHGKQADVERLLSKGQHLYKEKPSTQPVKRKLEDLRSEWEAVNHLLRELRTKQPDRAPGLSTTGASASQTVTLVTQSVVTKETVISKLEMPSSLLLEVPALADFNRAWTELTDWLSLLDRVIKSQRVMVGDLEDINEMIIKQKATLQDLEQRRPQLEELITAAQNLKNKTSNQEARTIITDRIERIQIQWDEVQEQLQNRRQQLNEMLKDSTQWLEAKEEAEQVIGQVRGKLDSWKEGPHTVDAIQKKITETKQLAKDLRQRQISVDVANDLALKLLRDYSADDTRKVHMITENINTSWGNIHKRVSEQEAALEETHRLLQQFPLDLEKFLSWITEAETTANVLQDASRKEKLLEDSRGVRELMKPWQDLQGEIETHTDIYHNLDENGQKILRSLEGSDEAPLLQRRLDNMNFKWSELQKKSLNIRSHLEASSDQWKRLHLSLQELLVWLQLKDDELSRQAPIGGDFPAVQKQNDIHRAFKRELKTKEPVIMSTLETVRIFLTEQPLEGLEKLYQEPRELPPEERAQNVTRLLRKQAEEVNAEWDKLNLRSADWQRKIDEALERLQELQEAADELDLKLRQAEVIKGSWQPVGDLLIDSLQDHLEKVKALRGEIAPLKENVNRVNDLAHQLTTLGIQLSPYNLSTLEDLNTRWRLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSESMGEEDLLSPPQDTSTGLEEVMEQLNNSFPSSRGRNAPGKPMREDTM

SEQ ID NO: 3 - protein, 75 aa, Artificial Sequence
  Synthetic
MLPRPPKVTKEEHFQLHHQMHYSQQITVSLAQGYERTSSPKPRFKSYAYTQAAYVTTSDPTRSPFPSQHLEAPED

SEQ ID NO: 4 - protein, 77 aa, Artificial Sequence
  Synthetic
MLPRTSSKVTREEHFQLHHQMHYSQQITVSLAQGYEQTSSSPKPRFKSYAFTQAAYVATSDSTQSPYPSQHLEAPRD

SEQ ID NO: 5 - protein, 72 aa, Artificial Sequence
  Synthetic
AHRDFGPASQHFLSTSVQGPWERAISPNKVPYYINHETQTTCWDHPKMTELYQSLADLNNVRFSAYRTAMKL

SEQ ID NO: 6 - protein, 72 aa, Artificial Sequence; identical to SEQ ID NO: 5
  Synthetic

SEQ ID NO: 7 - DNA, 3210 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaaacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtag

SEQ ID NO: 8 - protein, 1069 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHREISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 9 - DNA, 3219 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaaacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaacgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtag

SEQ ID NO: 10 - protein, 1072 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHREISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHERRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 11 - DNA, 3390 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaaacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaatctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtag

SEQ ID NO: 12 - protein, 1129 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHREISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHESVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 13 - DNA, 3375 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaaacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtag

SEQ ID NO: 14 - protein, 1124 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHREISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 15 - DNA, 3330 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaaacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaatctgtccagggtccctgggagagagccatctcgccaaacaaagtgccctactatatcaaccacgagactcaaacaacttgctgggaccatcccaaaatgacagagctctaccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtag

SEQ ID NO: 16 - protein, 1109 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHREISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHESVQGPWERAISPNKVPYYINHETQTTCWDHPKMTELYRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 17 - DNA, 3270 bp, Artificial Sequence
  Synthetic
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaaacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaacacgagactcaaacaacttgctgggaccatcccaaaatgacagagctctaccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtag

SEQ ID NO: 18 - protein, 1089 aa, Artificial Sequence
  Synthetic
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHREISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEHETQTTCWDHPKMTELYRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM